(19) United States
(12) Patent Application Publication
(10) Pub. No.: US 2010/0048460 A1
(43) Pub. Date: Feb. 25, 2010

(54) SELECTIVE VPAC2 RECEPTOR PEPTIDE AGONISTS

(76) Inventors: Lianshan Zhang, Carmel, IN (US); Jorge Alsina-Fernandez, Indianapolis, IN (US)

Correspondence Address: ELI LILLY & COMPANY, PATENT DIVISION, P.O. BOX 6288, INDIANAPOLIS, IN, 46206-6288, US

(21) Appl. No.: 12/278,035

(22) PCT Filed: Feb. 26, 2007
(86) PCT No.: PCT/US07/62783

§ 371 Date: Aug. 1, 2008

Related U.S. Application Data
(60) Provisional application No. 60/743,365, filed on Feb. 28, 2006.

(51) Int. Cl. A61K 38/16; C07K 14/00

(52) U.S. Cl. 514/9; 530/317

(57) ABSTRACT

The present invention encompasses peptides that selectively activate the VPAC2 receptor and are useful in the treatment of diabetes.

The present invention relates to selective VPAC2 receptor peptide agonists.

In particular, the present invention relates to selective VPAC2 receptor peptide agonists which are cyclic.

Type 2 diabetes, or non-insulin dependent diabetes mellitus (NIDDM), is the most common form of diabetes, affecting 90% of people with diabetes. With NIDDM, patients have impaired β-cell function resulting in insufficient insulin production and/or decreased insulin sensitivity. If NIDDM is not controlled, excess glucose accumulates in the blood, resulting in hyperglycemia. Over time, more serious complications may arise including renal dysfunction, cardiovascular problems, visual loss, lower limb ulceration, neuropathy, and ischemia. Treatments for NIDDM include improving diet, exercise, and weight control as well as using a variety of oral medications. Individuals with NIDDM can initially control their blood glucose levels by taking such oral medications. These medications, however, do not slow the progressive loss of β-cell function that occurs in NIDDM patients and, thus, are not sufficient to control blood glucose levels in the later stages of the disease. Also, treatment with currently available medications exposes NIDDM patients to potential side effects such as hypoglycemia, gastrointestinal problems, fluid retention, oedema, and/or weight gain.

Pituitary adenylate cyclase-activating peptide (PACAP) and vasoactive intestinal peptide (VIP) belong to the same family of peptides as secretin and glucagon. PACAP and VIP work through three G-protein-coupled receptors that exert their action through the cAMP-mediated and other Ca²⁺-mediated signal transduction pathways. These receptors are known as the PACAP-preferring type 1 (PAC 1) receptor (Isobe, et al., Regul. Pept., 110:213-217 (2003); Ogi, et al., Biochem. Biophys. Res. Commun., 196:1511-1521 (1993)) and the two VIP-shared type 2 receptors (VPAC1 and VPAC2) (Sherwood et al., Endocr. Rev., 21:619-670 (2000); Hammar et al., Pharmacol Rev, 50:265-270 (1998); Couvineau, et al., J. Biol. Chem., 278:24759-24766 (2003); Sreedharan, et al., Biochem. Biophys. Res. Commun., 193:546-553 (1993); Lutz, et al., FEBS Lett., 458: 197-203 (1999); Adamou, et al., Biochem. Biophys. Res. Commun., 209: 385-392 (1995)). A series of PACAP analogues is disclosed in U.S. Pat. No. 6,242,563 and WO 2000/05260.

PACAP has comparable activities towards all three receptors, whilst VIP selectively activates the two VPAC receptors (Tsutsumi et al., Diabetes, 51:1453-1460 (2002)). Both VIP (Eriksson et al., Peptides, 10: 481-484 (1989)) and PACAP (Filipsson et al., JCEM, 82:3093-3098 (1997)) have been shown to not only stimulate insulin secretion in man when given intravenously but also increase glucagon secretion and hepatic glucose output. As a consequence, PACAP or VIP stimulation generally does not result in a net improvement of glycemia. Activation of multiple receptors by PACAP or VIP also has broad physiological effects on nervous, endocrine, cardiovascular, reproductive, muscular, and immune systems (Gozes et al., Curr. Med. Chem., 6:1019-1034 (1999)). Furthermore, it appears that VIP-induced watery diarrhoea in rats is mediated by only one of the VPAC receptors, VPAC1 (Ito et al., Peptides, 22:1139-1151 (2001); Tsutsumi et al., Diabetes, 51:1453-1460 (2002)). In addition, the VPAC1 and PAC1 receptors are expressed on α-cells and hepatocytes and, thus, are most likely involved in the effects on hepatic glucose output.

Exendin-4 is found in the salivary excretions from the Gila Monster, Heloderma Suspectum, (Eng et al, J. Biol. Chem., 267(11):7402-7405 (1992)). It is a 39 amino acid peptide, which has glucose dependent insulin secretagogue activity. Particular PEGylated Exendin and Exendin agonist peptides are described in WO 2000/66629.

Information obtained from studying the structure and proteolytic cleavage of linear VIP analogues has been used in the synthesis and development of cyclic VIP analogues (Bolin et al., Biopolymers (Peptide Science), 37:57-66 (1995) and Bolin et al., Drug Design and Discovery, 13:107-114 (1996)). U.S. Pat. No. 5,677,419 and EP 0 536 741 (Hoffmann-La Roche Inc.) disclose a series of cyclised VIP analogues, which are useful for the treatment of asthma. A process for the synthesis of a cyclic VIP analogue from four protected peptides fragments is described in U.S. Pat. No. 6,080,837 (also, U.S. Pat. No. 6,316,593) and WO 97/29126 (Hoffmann-La Roche Inc.). One particular cyclic VIP analogue, identified as RO 15-1392, has been shown to be a selective VPAC2 receptor agonist (Bolin et al, J. Pharmacol. Exp. Ther., 281(2):629-633 (1997)). In addition, a cyclic VIP analogue was used as the starting point for the development of a VPAC2 receptor peptide antagonist (Moreno et al., Peptides, 21:1543-1549 (2000)).

Recent studies have shown that peptides selective for the VPAC2 receptor are able to stimulate insulin secretion from the pancreas without gastrointestinal (GI) side effects and without enhancing glucagon release and hepatic glucose output (Tsutsumi et al., Diabetes, 51:1453-1460 (2002)). Peptides selective for the VPAC2 receptor, were initially identified by modifying VIP and/or PACAP (See, for example, Xia et al., J Pharmacol Exp Ther., 281:629-633 (1997); Tsutsumi et al., Diabetes, 51:1453-1460 (2002); WO 01/23420; WO 2004/006839).

Many of the VPAC2 receptor peptide agonists reported to date have, however, less than desirable potency, selectivity, and stability profiles, which could impede their clinical viability. In addition, many of these peptides are not suitable for commercial candidates as a result of stability issues associated with the polypeptides in formulation, as well as issues with the short half-life of these polypeptides in vivo. There is, therefore, a need for new therapies, which overcome the problems associated with current medications for NIDDM.

The present invention seeks to provide improved compounds that are selective for the VPAC2 receptor and which induce insulin secretion from the pancreas only in the presence of high blood glucose levels. The compounds of the present invention are peptides, which are believed to also improve beta cell function. These peptides can have the physiological effect of inducing insulin secretion without GI side effects or a corresponding increase in hepatic glucose output and also generally have enhanced selectivity, potency, and/or in vivo stability of the peptide compared to known VPAC2 receptor peptide agonists.

The present invention particularly seeks to provide cyclic VPAC2 receptor peptide agonists, having increased selectivity, potency and/or stability compared to linear VPAC2 receptor peptide agonists.

According to a first aspect of the invention, there is provided a cyclic VPAC2 receptor peptide agonist comprising the amino acid sequence:

| Agonist # | SEQ ID NO: | Sequence |
|---|---|---|
| P403 | 1 | 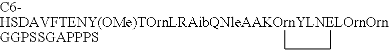 |

According to the second aspect of the present invention, there is provided a pharmaceutical composition comprising a cyclic VPAC2 receptor peptide agonist of the present invention and one or more pharmaceutically acceptable diluents, carriers and/or excipients.

According to a third aspect of the present invention, there is provided a cyclic VPAC2 receptor peptide agonist of the present invention for use as a medicament.

According to a fourth aspect of the present invention, there is provided a cyclic VPAC2 receptor peptide agonist of the present invention for use in the treatment of non-insulin-dependent diabetes or insulin-dependent diabetes, or for use in the suppression of food intake.

According to a fifth aspect of the present invention, there is provided the use of a cyclic VPAC2 receptor peptide agonist of the present invention for the manufacture of a medicament for the treatment of non-insulin-dependent diabetes or insulin-dependent diabetes, or for the suppression of food intake.

According to a further aspect of the present invention, there is provided a method of treating non-insulin-dependent diabetes or insulin-dependent diabetes, or of suppressing food intake in a patient in need thereof comprising administering an effective amount of a cyclic VPAC2 receptor peptide agonist of the present invention.

According to yet a further aspect of the present invention, there is provided a pharmaceutical composition containing a cyclic VPAC2 receptor peptide agonist of the present invention for treating non-insulin-dependent diabetes or insulin-dependent diabetes, or for suppressing food intake.

The VPAC2 receptor peptide agonists of the present invention have the advantage that they have enhanced selectivity, potency and/or stability over known VPAC2 receptor peptide agonists. The addition of the C-terminal sequence of Exendin-4, or a variant of this C-terminal sequence, as the c-capping sequence surprisingly increased the VPAC2 receptor selectivity as well as increasing proteolytic stability. In particular, cyclic VPAC2 receptor peptide agonists have restricted conformational mobility compared to linear VPAC2 receptor peptide agonists of small/medium size and for this reason cyclic peptides have a smaller number of allowed conformations compared with linear peptides. Constraining the conformational flexibility of linear peptides by cyclisation enhances receptor-binding affinity, increases selectivity and improves proteolytic stability and bioavailability compared with linear peptides.

Cyclic VPAC2 receptor peptide agonists of the present invention may be PEGylated. PEGylation is the covalent attachment of one or more molecules of polyethylene glycol (PEG), or a derivative thereof, to particular residues of a VPAC2 receptor peptide agonist. For example, a PEG molecule may be attached to a lysine amino acid in the peptide agonist.

The term “VPAC2” is used to refer to the particular receptor (Lutz, et al., FEBS Lett., 458: 197-203 (1999); Adamou, et al., Biochem. Biophys. Res. Commun., 209: 385-392 (1995)) that the agonists of the present invention activate. This term also is used to refer to the agonists of the present invention.

A “selective VPAC2 receptor peptide agonist” or a “VPAC2 receptor peptide agonist” of the present invention is a peptide that selectively activates the VPAC2 receptor to induce insulin secretion. Preferably, the sequence for a selective VPAC2 receptor peptide agonist has twenty-eight naturally occurring and/or non-naturally occurring amino acids and additionally comprises a C-terminal extension.

A “selective cyclic VPAC2 receptor peptide agonist” or a “cyclic VPAC2 receptor peptide agonist” is a selective VPAC2 receptor peptide agonist cyclised by means of a covalent bond linking the side chains of two amino acids in the peptide chain. The covalent bond may, for example, be a lactam bridge or a disulfide bridge.

The term “lactam bridge” as used herein means a covalent bond, in particular an amide bond, linking the side chain amino terminus of one amino acid in the peptide agonist to the side chain carboxy terminus of another amino acid in the peptide agonist. A lactam bridge may be formed by the covalent attachment of the side chain of a residue at Xaaₙ to the side chain of a residue at Xaaₙ₊₄, wherein n is 1 to 28. A lactam bridge may be formed by the covalent attachment of the side chain amino terminus of a Lys, Orn, or Dab residue to the side chain carboxy terminus of an Asp or Glu residue. P403 has a lactam bridge which is formed by the covalent attachment of the side chain amino terminus of the Orn residue at position 21 and the side chain carboxy terminus of the Glu residue at position 25.

The term “disulfide bridge” as used herein means a covalent bond linking a sulfur atom at the side chain terminus of one amino acid in the peptide agonist to a sulfur atom at the side chain terminus of another amino acid in the peptide agonist. A disulfide bridge may be formed by the covalent attachment of the side chain of a residue at Xaaₙ to the side chain of a residue at Xaaₙ₊₄, wherein n is 1 to 28. A disulfide bridge may be formed by the covalent attachment of the side chain of a Cys or hC residue to the side chain of another Cys or hC residue.

Selective cyclic VPAC2 receptor peptide agonists of the present invention have a C-terminal extension. A “C-terminal extension” may comprise a sequence having from one to thirteen naturally occurring or non-naturally occurring amino acids linked to the C-terminus of the peptide sequence at the N-terminus of the C-terminal extension via a peptide bond. Any Cys, Lys, K(W), or K(CO(CH₂)₂SH) residues in the C-terminal extension may be covalently attached to a PEG molecule, and/or the carboxy-terminal amino acid of the C-terminal extension may be covalently attached to a PEG molecule. The C-terminal extension of P403 is GGPSSGAPPPS (SEQ ID NO: 7).

As used herein, the term “linked to” with reference to the term C-terminal extension, includes the addition or attachment of amino acids or chemical groups directly to the C-terminus of the peptide sequence.

The selective cyclic VPAC2 receptor peptide agonists of the present invention have an N-terminal modification. The N-terminal modification of P403 is the addition of a hexanoyl group. Other examples of N-terminal modifications are described below.

The term “N-terminal modification” as used herein includes the addition or attachment of amino acids or chemical groups directly to the N-terminus of a peptide and the formation of chemical groups, which incorporate the nitrogen at the N-terminus of a peptide.

An N-terminal modification may comprise the addition of one or more naturally occurring or non-naturally occurring amino acids to the VPAC2 receptor peptide agonist sequence, preferably there are not more than ten amino acids, with one amino acid being more preferred. Naturally occurring amino acids which may be added to the N-terminus include methionine and isoleucine. A modified amino acid added to the N-terminus may be D-histidine. Alternatively, the following amino acids may be added to the N-terminus: SEQ ID NO: 5 Ser-Trp-Cys-Glu-Pro-Gly-Trp-Cys-Arg, wherein the Arg is linked to the N-terminus of the peptide agonist. Preferably, any amino acids added to the N-terminus are linked to the N-terminus by a peptide bond.

The term “linked to” as used herein, with reference to the term N-terminal modification, includes the addition or attachment of amino acids or chemical groups directly to the N-terminus of the VPAC2 receptor agonist. The addition of the above N-terminal modifications may be achieved under normal coupling conditions for peptide bond formation.

The N-terminus of the peptide agonist may also be modified by the addition of an alkyl group (R), preferably a C₁-C₁₆ alkyl group, to form (R)NH—.

Alternatively, the N-terminus of the peptide agonist may be modified by the addition of a group of the formula —C(O)R¹ to form an amide of the formula R¹C(O)NH—. The addition of a group of the formula —C(O)R¹ may be achieved by reaction with an organic acid of the formula R¹COOH. Modification of the N-terminus of an amino acid sequence using acylation is demonstrated in the art (e.g. Gozes et al., J. Pharmacol Exp Ther, 273:161-167 (1995)). Addition of a group of the formula —C(O)R¹ may result in the formation of a urea group (see WO 01/23240, WO 2004/006839) or a carbamate group at the N-terminus. Also, the N-terminus may be modified by the addition of pyroglutamic acid, or 6-aminohexanoic acid.

The N-terminus of the peptide agonist may be modified by the addition of a group of the formula —SO₂R⁵, to form a sulfonamide group at the N-terminus.

The N-terminus of the peptide agonist may also be modified by reacting with succinic anhydride to form a succinimide group at the N-terminus. The succinimide group incorporates the nitrogen at the N-terminus of the peptide.

The N-terminus may alternatively be modified by the addition of methionine sulfoxide, biotinyl-6-aminohexanoic acid, or —C(═NH)—NH₂. The addition of —C(═NH)—NH₂ is a guanidation modification, where the terminal NH₂ of the N-terminal amino acid becomes —NH—C(═NH)—NH₂.

Most of the sequences of the present invention, including the N-terminal modifications and the C-terminal extensions contain the standard single letter or three letter codes for the twenty naturally occurring amino acids. The other codes used herein are defined as follows:

C6=hexanoyl

Aib=amino isobutyric acid

OMe=methoxy

Nle=Nor-leucine

Orn=ornithine

K(CO(CH₂)₂SH)=ε-(3′-mercaptopropionyl)-lysine

K(W)=ε-(L-tryptophyl)-lysine

Dab=diaminobutyric acid

hC=homocysteine

PEG=polyethylene glycol

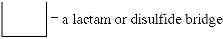

VIP naturally occurs as a single sequence having 28 amino acids. However, PACAP exists as either a 38 amino acid peptide (PACAP-38) or as a 27 amino acid peptide (PACAP-27) with an amidated carboxyl (Miyata, et al., Biochem Biophys Res Commun, 170:643-648 (1990)). The sequences for VIP, PACAP-27, and PACAP-38 are as follows:

| | Seq.ID | |
|---|---|---|
| Peptide | # | Sequence |
| VIP | SEQ ID | HSDAVFTDNYTRLRKQMAVKKYLNSILN |
| | NO: 2 | |
| PACAP-27 | SEQ ID | HSDGIFTDSYSRYRKQMAVKKYLAAVL-NH₂ |
| | NO: 3 | |
| PACAP-38 | SEQ ID | HSDGIFTDSYSRYRKQMAVKKYLAAVLGKRYQRVKN |
| | NO: 4 | K-NH₂ |

The term “naturally occurring amino acid” as used herein means the twenty amino acids coded for by the human genetic code (i.e. the twenty standard amino acids). These twenty amino acids are: Alanine, Arginine, Asparagine, Aspartic Acid, Cysteine, Glutamine, Glutamic Acid, Glycine, Histidine, Isoleucine, Leucine, Lysine, Methionine, Phenylalanine, Proline, Serine, Threonine, Tryptophan, Tyrosine and Valine.

Examples of “non-naturally occurring amino acids” include both synthetic amino acids and those modified by the body. These include D-amino acids, arginine-like amino acids (e.g., homoarginine), and other amino acids having an extra methylene in the side chain (“homo” amino acids), and modified amino acids (e.g. norleucine, lysine (isopropyl)—wherein the side chain amine of lysine is modified by an isopropyl group). Also included are amino acids such as ornithine, amino isobutyric acid and 2-aminobutanoic acid.

“Selective” as used herein refers to a VPAC2 receptor peptide agonist with increased selectivity for the VPAC2 receptor compared to other known receptors. The degree of selectivity is determined by a ratio of VPAC2 receptor binding affinity to VPAC1 receptor binding affinity or by a ratio of VPAC2 receptor binding affinity to PAC1 receptor binding affinity. Binding affinity is determined as described below in Example 4.

“Insulinotropic activity” refers to the ability to stimulate insulin secretion in response to elevated glucose levels, thereby causing glucose uptake by cells and decreased plasma glucose levels. Insulinotropic activity can be assessed by methods known in the art, including using experiments that measure VPAC2 receptor binding activity or receptor activation (e.g. insulin secretion by insulinoma cell lines or islets, intravenous glucose tolerance test (IVGTT), intraperitoneal glucose tolerance test (IPGTT), and oral glucose tolerance test (OGTT)). Insulinotropic activity is routinely measured in humans by measuring insulin levels or C-peptide levels. Selective VPAC2 receptor peptide agonists of the present invention have insulinotropic activity.

“In vitro potency” as used herein is the measure of the ability of a peptide to activate the VPAC2 receptor in a cell-based assay. In vitro potency is expressed as the “EC₅₀” which is the effective concentration of compound that results in a 50% of maximum increase in activity in a single dose-response experiment. For the purposes of the present invention, in vitro potency is determined using two different assays: DiscoveRx and Alpha Screen. See Examples 3 and 5 for further details of these assays. Whilst these assays are performed in different ways, the results demonstrate a general correlation between the two assays.

The term “plasma half-life” refers to the time in which half of the relevant molecules circulate in the plasma prior to being cleared. An alternatively used term is “elimination half-life.” The term “extended” or “longer” used in the context of plasma half-life or elimination half-life indicates there is a statistically significant increase in the half-life of a PEGylated VPAC2 receptor peptide agonist relative to that of the reference molecule (e.g., the non-PEGylated form of the peptide or the native peptide) as determined under comparable conditions. The person skilled in the art appreciates that half-life is a derived parameter that changes as a function of both clearance and volume of distribution.

Clearance is the measure of the body's ability to eliminate a drug. As clearance decreases due, for example, to modifications to a drug, half-life would be expected to increase. However, this reciprocal relationship is exact only when there is no change in the volume of distribution. A useful approximate relationship between the terminal log-linear half-life (t_{1/2}), clearance (C), and volume of distribution (V) is given by the equation: t_{1/2}≈0.693 (V/C). Clearance does not indicate how much drug is being removed but, rather, the volume of biological fluid such as blood or plasma that would have to be completely freed of drug to account for the elimination. Clearance is expressed as a volume per unit of time.

“Percent (%) sequence identity” as used herein is used to denote sequences which when aligned have similar (identical or conservatively replaced) amino acids in like positions or regions, where identical or conservatively replaced amino acids are those which do not alter the activity or function of the protein as compared to the starting protein. For example, two amino acid sequences with at least 85% identity to each other have at least 85% similar (identical or conservatively replaced residues) in a like position when aligned optimally allowing for up to 3 gaps, with the proviso that in respect of the gaps a total of not more than 15 amino acid residues is affected.

The reference peptide used for the percentage sequence identity calculations herein is:

| Agonist # | SEQ ID NO: | Sequence |
|---|---|---|
| P57 | 6 | 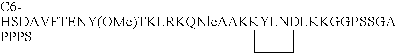 |

Percent sequence identity may be calculated by determining the number of residues that differ between a peptide encompassed by the present invention and a reference peptide such as P57 (SEQ ID NO: 6), taking that number and dividing it by the number of amino acids in the reference peptide (e.g. 39 amino acids for P57), multiplying the result by 100, and subtracting that resulting number from 100. For example, a sequence having 39 amino acids with four amino acids that are different from P57 would have a percent (%) sequence identity of 90% (e.g. 100−((4/39)×100)). For a sequence that is longer than 39 amino acids, the number of residues that differ from the P57 sequence will include the additional amino acids over 39 for purposes of the aforementioned calculation. For example, a sequence having 41 amino acids, with four amino acids different from the 39 amino acids in the P57 sequence and with two additional amino acids at the carboxy terminus which are not present in the P57 sequence, would have a total of six amino acids that differ from P57. Thus, this sequence would have a percent (%) sequence identity of 84% (e.g. 100−((6/39)×100)). The degree of sequence identity may be determined using methods well known in the art (see, for example, Wilbur, W. J. and Lipman, D. J., Proc. Natl. Acad. Sci. USA 80:726-730 (1983) and Myers E. and Miller W., Comput. Appl. Biosci. 4:11-17 (1988)). One program which may be used in determining the degree of similarity is the MegAlign Lipman-Pearson one pair method (using default parameters) which can be obtained from DNAstar Inc, 1128, Selfpark Street, Madison, Wis., 53715, USA as part of the Lasergene system. Another program, which may be used, is Clustal W. This is a multiple sequence alignment package developed by Thompson et al (Nucleic Acids Research, 22(22):4673-4680(1994)) for DNA or protein sequences. This tool is useful for performing cross-species comparisons of related sequences and viewing sequence conservation. Clustal W is a general purpose multiple sequence alignment program for DNA or proteins. It produces biologically meaningful multiple sequence alignments of divergent sequences. It calculates the best match for the selected sequences, and lines them up so that the identities, similarities and differences can be seen. Evolutionary relationships can be seen via viewing Cladograms or Phylograms.

A selective cyclic VPAC2 receptor peptide agonist is selective for the VPAC2 receptor and may have a sequence identity in the range of 60% to 70%, 60% to 65%, 65% to 70%, 70% to 80%, 70% to 75%, 75% to 80%, 80% to 90%, 80% to 85%, 85%, to 90%, 90% to 97%, 90% to 95%, or 95% to 97%, with P57 (SEQ ID NO: 6). P403 has a sequence identity of 85% with P57.

The term “PEG” as used herein means a polyethylene glycol molecule. In its typical form, PEG is a linear polymer with terminal hydroxyl groups and has the formula HO—CH₂CH₂—(CH₂CH₂O)n-CH₂CH₂—OH, where n is from about 8 to about 4000. The terminal hydrogen may be substituted with a protective group such as an alkyl or alkanol group. Preferably, PEG has at least one hydroxy group, more preferably it is a terminal hydroxy group. It is this hydroxy group which is preferably activated to react with the peptide. Numerous derivatives of PEG exist in the art. (See, e.g., U.S. Pat. Nos. 5,445,090; 5,900,461; 5,932,462; 6,436,386; 6,448,369; 6,437,025; 6,448,369; 6,495,659; 6,515,100 and 6,514,491 and Zalipsky, S. Bioconjugate Chem. 6:150-165, 1995). The molecular weight of the PEG molecule is preferably from 500-100,000 daltons. PEG may be linear or branched and PEGylated VPAC2 receptor peptide agonists may have one, two or three PEG molecules attached to the peptide. It is more preferable that there be one or two PEG molecules per PEGylated VPAC2 receptor peptide agonist. It is further contemplated that both ends of the PEG molecule may be homo- or hetero-functionalized for crosslinking two or more VPAC2 receptor peptide agonists together.

In the present invention, a PEG molecule may be covalently attached to the Lys residue of P403. Any Lys residue in a peptide agonist may be substituted for a K(W) or K(CO(CH₂)₂SH), which may then be PEGylated. K(W) is a Trp residue coupled to the side chain of a Lys residue and it is PEGylated by covalently attaching a PEG molecule to the Trp residue. A K(CO(CH₂)₂SH) group is PEGylated to form K(CO(CH₂)₂S-PEG).

The term “PEGylation” as used herein means the covalent attachment of one or more PEG molecules as described above to the VPAC2 receptor peptide agonist.

The region of wild-type VIP from aspartic acid at position 8 to isoleucine at position 26 has an alpha-helix structure. Increasing the helical content of a peptide enhances potency and selectivity whilst at the same time improving protection from enzymatic degradation. The use of a C-terminal extension, such as an Exendin-4 extension, may enhance the helicity of the peptide. In addition, the introduction of a covalent bond, for example a lactam bridge, linking the side chains of two amino acids on the surface of the helix, also enhances the helicity of the peptide.

PEGylation of proteins may overcome many of the pharmacological and toxicological/immunological problems associated with using peptides or proteins as therapeutics. However, for any individual peptide it is uncertain whether the PEGylated form of the peptide will have significant loss in bioactivity as compared to the unPEGylated form of the peptide.

The bioactivity of PEGylated proteins can be affected by factors such as: i) the size of the PEG molecule; ii) the particular sites of attachment; iii) the degree of modification; iv) adverse coupling conditions; v) whether a linker is used for attachment or whether the polymer is directly attached; vi) generation of harmful co-products; vii) damage inflicted by the activated polymer; or viii) retention of charge. Work performed on the PEGylation of cytokines, for example, shows the effect PEGylation may have. Depending on the coupling reaction used, polymer modification of cytokines has resulted in dramatic reductions in bioactivity [Francis, G. E., et al., (1998) PEGylation of cytokines and other therapeutic proteins and peptides: the importance of biological optimization of coupling techniques, Intl. J. Hem. 68:1-18]. Maintaining the bioactivity of PEGylated peptides is even more problematic than for proteins. As peptides are smaller than proteins, modification by PEGylation may potentially have a greater effect on bioactivity.

The VPAC2 receptor peptide agonists of the present invention may be modified by the covalent attachment of one molecule of a polyethylene glycol (PEG) and may have improved pharmacokinetic profiles due to slower proteolytic degradation and renal clearance. Attachment of a PEG molecule (PEGylation) will increase the apparent size of the VPAC2 receptor peptide agonists, thus reducing renal filtration and altering biodistribution. PEGylation can shield antigenic epitopes of the VPAC2 receptor peptide agonists, thus reducing reticuloendothelial clearance and recognition by the immune system and also reducing degradation by proteolytic enzymes, such as DPP-IV.

Covalent attachment of a molecule of PEG to a small, biologically active VPAC2 receptor peptide agonist poses the risk of adversely affecting the agonist, for example, by destabilising the inherent secondary structure and bioactive conformation and reducing bioactivity, so as to make the agonist unsuitable for use as a therapeutic. However, PEGylation of a VPAC2 receptor peptide agonist may surprisingly result in a biologically active, PEGylated VPAC2 receptor peptide agonist with an extended half-life and reduced clearance when compared to that of non-PEGylated VPAC2 receptor peptide agonists.

In order to determine the potential PEGylation sites in a VPAC2 receptor peptide agonist, serine scanning may be conducted. A Ser residue is substituted at a particular position in the peptide and the Ser-modified peptide is tested for potency and selectivity. If the Ser substitution has minimal impact on potency and the Ser-modified peptide is selective for the VPAC2 receptor, the Ser residue is then substituted for a Cys or Lys residue, which serves as a direct or indirect PEGylation site. Indirect PEGylation of a residue is the PEGylation of a chemical group or residue which is bonded to the PEGylation site residue. Indirect PEGylation of Lys includes PEGylation of K(W) and K(CO(CH₂)₂SH).

VPAC2 receptor peptide agonists of the present invention may be covalently attached to one molecule of polyethylene glycol (PEG), or a derivative thereof. PEGylation can enhance the half-life of the selective VPAC2 receptor peptide agonists, resulting in PEGylated VPAC2 receptor peptide agonists with an elimination half-life of at least one hour, preferably at least 3, 5, 7, 10, 15, 20, or 24 hours and most preferably at least 48 hours. PEGylated VPAC2 receptor peptide agonists preferably have a clearance value of 200 ml/h/kg or less, more preferably 180, 150, 120, 100, 80, 60 ml/h/kg or less and most preferably less than 50, 40 or 20 ml/h/kg.

The present invention encompasses the discovery that specific amino acids added to the C-terminus of a peptide sequence for a VPAC2 receptor peptide agonist may protect the peptide as well as may enhance activity, selectivity, and/or potency. For example, these C-terminal extensions may stabilize the helical structure of the peptide and stabilize sites located near to the C-terminus, which are prone to enzymatic cleavage. Furthermore, many of the C-terminally extended peptides disclosed herein may be more selective for the VPAC2 receptor and can be more potent than VIP, PACAP, and other known VPAC2 receptor peptide agonists. An example of a preferred C-terminal extension is the extension peptide of Exendin-4; GGPSSGAPPPS. This Exendin-4 C-terminal extension is the C-terminal extension of P403. Exendin-4 is found in the salivary excretions from the Gila Monster, Heloderma Suspectum, (Eng et al., J. Biol. Chem., 267(11):7402-7405 (1992)). Other examples of C-terminal extensions are the C-terminal sequences of helodermin and helospectin. Helodermin and helospectin are also found in the salivary excretions of the Gila Monster.

It has, furthermore, been discovered that modification of the N-terminus of the VPAC2 receptor peptide agonist may enhance potency and/or provide stability against DPP-IV cleavage.

VIP and some known VPAC2 receptor peptide agonists are susceptible to cleavage by various enzymes and, thus, have a short in vivo half-life. Various enzymatic cleavage sites in the VPAC2 receptor peptide agonists are discussed below. The cleavage sites are discussed relative to the amino acid positions in VIP (SEQ ID NO: 2), and are applicable to the sequences noted herein.

Cleavage of the peptide agonist by the enzyme dipeptidyl-peptidase-IV (DPP-IV) occurs between position 2 (serine in VIP) and position 3 (aspartic acid in VIP). The compounds of the present invention may be rendered more stable to DPP-IV cleavage in this region by the addition of a N-terminal modification. Examples of N-terminal modifications that may improve stability against DPP-IV cleavage include the addition of acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid, or —C(═NH₂)—NH₂.

There are chymotrypsin cleavage sites in wild-type VIP between the amino acids 10 and 11 (tyrosine and threonine) and those at 22 and 23 (tyrosine and leucine). Substituting Tyr(OMe) for tyrosine may increase stability at the 10-11 site. A lactam bridge, for example, linking the side chains of the amino acids at positions 21 and 25 may protect the 22-23 site from cleavage.

There is a trypsin cleavage site between the amino acids at positions 12 and 13 of wild-type VIP. Certain amino acids render the peptide less susceptible to cleavage at this site, for example, ornithine at position 12.

In wild-type VIP, and in numerous VPAC2 receptor peptide agonists known in the art, there are cleavage sites between the basic amino acids at positions 14 and 15 and between those at positions 20 and 21. The selective cyclic VPAC2 receptor peptide agonists of the present invention may have improved proteolytic stability in-vivo due to substitutions at these sites. The preferred substitutions at these sites are those which render the peptide less susceptible to cleavage by trypsin-like enzymes, including trypsin. For example, amino isobutyric acid at position 15 and ornithine at position 21 are preferred substitutions which may lead to improved stability.

There is also a cleavage site between the amino acids at positions 25 and 26 of wild type VIP.

The region of the VPAC2 receptor peptide agonist encompassing the amino acids at positions 27, 28 and 29 is also susceptible to enzyme cleavage. The addition of a C-terminal extension may render the peptide agonist more stable against neuroendopeptidase (NEP), and it may also increase selectivity for the VPAC2 receptor. This region may also be attacked by trypsin-like enzymes. If that occurs, the peptide agonist may lose its C-terminal extension with the additional carboxypeptidase activity leading to an inactive form of the peptide. Resistance to cleavage in this region may be increased by substituting the amino acid at position 27 and/or 28 with ornithine.

In addition to selective cyclic VPAC2 receptor peptide agonists with resistance to cleavage by various peptidases, the selective cyclic VPAC2 peptide receptor agonists of the present invention may also encompass peptides with enhanced selectivity for the VPAC2 receptor, increased potency, and/or increased stability compared with some peptides known in the art.

Preferably, the selective cyclic VPAC2 receptor peptide agonists of the present invention have an EC₅₀ value less than 2 nM. More preferably, the EC₅₀ value is less than 1 nM. Even more preferably, the EC₅₀ value is less than 0.5 nM. Still more preferably, the EC₅₀ value is less than 0.1 nM.

Preferably, the agonists of the present invention have a selectivity ratio where the affinity for the VPAC2 receptor is at least 50 times greater than for the VPAC1 and/or for PAC I receptors. More preferably, this affinity is at least 100 times greater for VPAC2 than for VPAC1 and/or for PAC1. Even more preferably, the affinity is at least 200 times greater for VPAC2 than for VPAC1 and/or for PAC1. Still more preferably, the affinity is at least 500 times greater for VPAC2 than for VPAC1 and/or for PAC1. Yet more preferably, the ratio is at least 1000 times greater for VPAC2 than for VPAC1 and/or for PAC1.

As used herein, “selective cyclic VPAC2 receptor peptide agonists” also include pharmaceutically acceptable salts of the agonists described herein. A selective VPAC2 receptor peptide agonist of this invention can possess a sufficiently acidic, a sufficiently basic, or both functional groups, and accordingly react with any of a number of inorganic bases, and inorganic and organic acids, to form a salt. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, trifluoroacetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The selective cyclic VPAC2 receptor peptide agonists of the present invention are preferably formulated as pharmaceutical compositions. Standard pharmaceutical formulation techniques may be employed such as those described in Remington's Pharmaceutical Sciences, Mack Publishing Company, Easton, Pa. The selective VPAC2 receptor peptide agonists of the present invention may be formulated for administration through the buccal, topical, oral, transdermal, nasal, or pulmonary route, or for parenteral administration.

Parenteral administration can include, for example, systemic administration, such as by intramuscular, intravenous, subcutaneous, intradermal, or intraperitoneal injection. The selective cyclic VPAC2 receptor peptide agonists can be administered to the subject in conjunction with an acceptable pharmaceutical carrier, diluent, or excipient as part of a pharmaceutical composition for treating NIDDM, or the disorders discussed below. The pharmaceutical composition can be a solution or, if administered parenterally, a suspension of the cyclic VPAC2 receptor peptide agonist or a suspension of the cyclic VPAC2 receptor peptide agonist complexed with a divalent metal cation such as zinc. Suitable pharmaceutical carriers may contain inert ingredients which do not interact with the peptide or peptide derivative. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Some examples of suitable excipients include lactose, dextrose, sucrose, trehalose, sorbitol, and mannitol.

The cyclic VPAC2 receptor peptide agonists of the invention may be formulated for administration such that blood plasma levels are maintained in the efficacious range for extended time periods. The main barrier to effective oral peptide drug delivery is poor bioavailability due to degradation of peptides by acids and enzymes, poor absorption through epithelial membranes, and transition of peptides to an insoluble form after exposure to the acidic pH environment in the digestive tract. Oral delivery systems for peptides such as those encompassed by the present invention are known in the art. For example, cyclic VPAC2 receptor peptide agonists can be encapsulated using microspheres and then delivered orally. For example, cyclic VPAC2 receptor peptide agonists can be encapsulated into microspheres composed of a commercially available, biocompatible, biodegradable polymer, poly(lactide-co-glycolide)-COOH and olive oil as a filler (see Joseph, et al. Diabetologia 43:1319-1328 (2000)). Other types of microsphere technology is also available commercially such as Medisorb® and Prolease® biodegradable polymers from Alkermes. Medisorb® polymers can be produced with any of the lactide isomers. Lactide:glycolide ratios can be varied between 0:100 and 100:0 allowing for a broad range of polymer properties. This allows for the design of delivery systems and implantable devices with resorption times ranging from weeks to months. Emisphere has also published numerous articles discussing oral delivery technology for peptides and proteins. For example, see WO 95/28838 by Leone-bay et al. which discloses specific carriers comprised of modified amino acids to facilitate absorption.

The selective cyclic VPAC2 receptor peptide agonists described herein can be used to treat subjects with a wide variety of diseases and conditions. Agonists encompassed by the present invention exert their biological effects by acting at a receptor referred to as the VPAC2 receptor. Subjects with diseases and/or conditions that respond favourably to VPAC2 receptor stimulation or to the administration of VPAC2 receptor peptide agonists can therefore be treated with the cyclic VPAC2 agonists of the present invention. These subjects are said to “be in need of treatment with VPAC2 agonists” or “in need of VPAC2 receptor stimulation”.

The selective cyclic VPAC2 receptor peptide agonists of the present invention may be employed to treat diabetes, including both type 1 and type 2 diabetes (non-insulin dependent diabetes mellitus or NIDDM). The agonists may also be used to treat subjects requiring prophylactic treatment with a VPAC2 receptor agonist, e.g., subjects at risk for developing NIDDM. Such treatment may also delay the onset of diabetes and diabetic complications. Additional subjects which may be treated with the agonists of the present invention include those with impaired glucose tolerance (IGT) (Expert Committee on Classification of Diabetes Mellitus, Diabetes Care 22 (Supp. 1):S5, 1999), or impaired fasting glucose (IFG) (Charles, et al., Diabetes 40:796, 1991), subjects whose body weight is about 25% above normal body weight for the subject's height and body build, subjects having one or more parents with NIDDM, subjects who have had gestational diabetes, and subjects with metabolic disorders such as those resulting from decreased endogenous insulin secretion. The selective cyclic VPAC2 receptor peptide agonists may be used to prevent subjects with impaired glucose tolerance from proceeding to develop NIDDM, prevent pancreatic β-cell deterioration, induce β-cell proliferation, improve β-cell function, activate dormant β-cells, differentiate cells into β-cells, stimulate β-cell replication, and inhibit β-cell apoptosis. Other diseases and conditions that may be treated or prevented using agonists of the invention in methods of the invention include: Maturity-Onset Diabetes of the Young (MODY) (Herman, et al., Diabetes 43:40, 1994); Latent Autoimmune Diabetes Adult (LADA) (Zimmet, et al., Diabetes Med. 11:299, 1994); gestational diabetes (Metzger, Diabetes, 40:197, 1991); metabolic syndrome X, dyslipidemia, hyperglycemia, hyperinsulinemia, hypertriglyceridemia, and insulin resistance.

The selective cyclic VPAC2 receptor peptide agonists of the invention may also be used in methods of the invention to treat secondary causes of diabetes (Expert Committee on Classification of Diabetes Mellitus, Diabetes Care 22 (Supp. 1):S5, 1999). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenytoin, thyroid hormone, β-adrenergic agents, α-interferon and drugs used to treat HIV infection.

The selective cyclic VPAC2 receptor peptide agonists of the present invention may be effective in the suppression of food intake and the treatment of obesity.

The selective cyclic VPAC2 receptor peptide agonists of the present invention may also be effective in the prevention or treatment of such disorders as atherosclerotic disease hyperlipidemia, hypercholesteremia, low HDL levels, hypertension, primary pulmonary hypertension, cardiovascular disease (including atherosclerosis, coronary heart disease and coronary artery disease), cerebrovascular disease and peripheral vessel disease; and for the treatment of lupus, polycystic ovary syndrome, carcinogenesis, and hyperplasia, male and female reproduction problems, sexual disorders, ulcers, sleep disorders, disorders of lipid and carbohydrate metabolism, circadian dysfunction, growth disorders, disorders of energy homeostasis, immune diseases including autoimmune diseases (e.g., systemic lupus erythematosus), as well as acute and chronic inflammatory diseases, rheumatoid arthritis, and septic shock.

The selective cyclic VPAC2 receptor peptide agonists of the present invention may also be useful for treating physiological disorders related to, for example, cell differentiation to produce lipid accumulating cells, regulation of insulin sensitivity and blood glucose levels, which are involved in, for example, abnormal pancreatic β-cell function, insulin secreting tumors and/or autoimmune hypoglycemia due to autoantibodies to insulin, autoantibodies to the insulin receptor, or autoantibodies that are stimulatory to pancreatic β-cells, macrophage differentiation which leads to the formation of atherosclerotic plaques, inflammatory response, carcinogenesis, hyperplasia, adipocyte gene expression, adipocyte differentiation, reduction in the pancreatic β-cell mass, insulin secretion, tissue sensitivity to insulin, liposarcoma cell growth, polycystic ovarian disease, chronic anovulation, hyperandrogenism, progesterone production, steroidogenesis, redox potential and oxidative stress in cells, nitric oxide synthase (NOS) production, increased gamma glutamyl transpeptidase, catalase, plasma triglycerides, HDL, and LDL cholesterol levels, and the like.

In addition, the selective cyclic VPAC2 receptor peptide agonists of the invention may be used for treatment of asthma (Bolin, et al., Biopolymer 37:57-66 (1995); U.S. Pat. No. 5,677,419; showing that polypeptide R3PO is active in relaxing guinea pig tracheal smooth muscle); hypotension induction (VIP induces hypotension, tachycardia, and facial flushing in asthmatic patients (Morice, et al., Peptides 7:279-280 (1986); Morice, et al., Lancet 2:1225-1227 (1983)); for the treatment of male reproduction problems (Siow, et al., Arch. Androl. 43(1):67-71 (1999)); as an anti-apoptosis/neuroprotective agent (Brenneman, et al., Ann. N.Y. Acad. Sci. 865:207-12 (1998)); for cardioprotection during ischemic events (Kalfin, et al., J. Pharmacol. Exp. Ther. 1268(2):952-8 (1994); Das, et al., Ann. N.Y. Acad. Sci. 865:297-308 (1998)); for manipulation of the circadian clock and its associated disorders (Hamar, et al., Cell 109:497-508 (2002); Shen, et al., Proc. Natl. Acad. Sci. 97:11575-80, (2000)); as an anti-ulcer agent (Tuncel, et al., Ann. N.Y. Acad. Sci. 865:309-22, (1998)); and as a treatment for AIDS (Branch, et al., Blood 106: Abstract 1427, (2005)).

An “effective amount” of a selective cyclic VPAC2 receptor peptide agonist is the quantity that results in a desired therapeutic and/or prophylactic effect without causing unacceptable side effects when administered to a subject in need of VPAC2 receptor stimulation. A “desired therapeutic effect” includes one or more of the following: 1) an amelioration of the symptom(s) associated with the disease or condition; 2) a delay in the onset of symptoms associated with the disease or condition; 3) increased longevity compared with the absence of the treatment; and 4) greater quality of life compared with the absence of the treatment. For example, an “effective amount” of a cyclic VPAC2 agonist for the treatment of NIDDM is the quantity that would result in greater control of blood glucose concentration than in the absence of treatment, thereby resulting in a delay in the onset of diabetic complications such as retinopathy, neuropathy, or kidney disease. An “effective amount” of a selective cyclic VPAC2 receptor peptide agonist for the prevention of NIDDM is the quantity that would delay, compared with the absence of treatment, the onset of elevated blood glucose levels that require treatment with anti-hypoglycemic drugs such as sulfonylureas, thiazolidinediones, insulin, and/or bisguanidines.

An “effective amount” of the selective cyclic VPAC2 receptor peptide agonist administered to a subject will also depend on the type and severity of the disease and on the characteristics of the subject, such as general health, age, sex, body weight and tolerance to drugs. The dose of selective cyclic VPAC2 peptide receptor agonist effective to normalize a patient's blood glucose will depend on a number of factors, among which are included, without limitation, the subject's sex, weight and age, the severity of inability to regulate blood glucose, the route of administration and bioavailability, the pharmacokinetic profile of the peptide, the potency, and the formulation.

A typical dose range for the selective cyclic VPAC2 receptor peptide agonists of the present invention will range from about 1 μg per day to about 5000 μg per day. Preferably, the dose ranges from about 1 μg per day to about 2500 μg per day, more preferably from about 1 μg per day to about 1000 μg per day. Even more preferably, the dose ranges from about 5 μg per day to about 100 μg per day. A further preferred dose range is from about 10 μg per day to about 50 μg per day. Most preferably, the dose is about 20 μg per day.

A “subject” is a mammal, preferably a human, but can also be an animal, e.g., companion animals (e.g., dogs, cats, and the like), farm animals (e.g., cows, sheep, pigs, horses, and the like) and laboratory animals (e.g., rats, mice, guinea pigs, and the like).

The selective cyclic VPAC2 receptor peptide agonists of the present invention can be prepared by using standard methods of solid-phase peptide synthesis techniques. Peptide synthesizers are commercially available from, for example, Rainin-PTI Symphony Peptide Synthesizer (Tucson, Ariz.). Reagents for solid phase synthesis are commercially available, for example, from Glycopep (Chicago, Ill.). Solid phase peptide synthesizers can be used according to manufacturers instructions for blocking interfering groups, protecting the amino acid to be reacted, coupling, decoupling, and capping of unreacted amino acids.

Typically, an α-N-protected amino acid and the N-terminal amino acid on the growing peptide chain on a resin is coupled at room temperature in an inert solvent such as dimethylformamide, N-methylpyrrolidone or methylene chloride in the presence of coupling agents such as dicyclohexylcarbodiimide and 1-hydroxybenzotriazole and a base such as diisopropylethylamine. The α-N-protecting group is removed from the resulting peptide resin using a reagent such as trifluoroacetic acid or piperidine, and the coupling reaction repeated with the next desired N-protected amino acid to be added to the peptide chain. Suitable amine protecting groups are well known in the art and are described, for example, in Green and Wuts, “Protecting Groups in Organic Synthesis”, John Wiley and Sons, 1991. Examples include t-butyloxycarbonyl (tBoc) and fluorenylmethoxycarbonyl (Fmoc).

The selective VPAC2 receptor peptide agonists may also be synthesized using standard automated solid-phase synthesis protocols using t-butoxycarbonyl- or fluorenylmethoxycarbonyl-alpha-amino acids with appropriate side-chain protection. After completion of synthesis, peptides are cleaved from the solid-phase support with simultaneous side-chain deprotection using standard hydrogen fluoride methods or trifluoroacetic acid (TFA). Crude peptides are then further purified using Reversed-Phase Chromatography on Vydac C18 columns using acetonitrile gradients in 0.1% TFA. To remove acetonitrile, peptides are lyophilized from a solution containing 0.1% TFA, acetonitrile and water. Purity can be verified by analytical reversed phase chromatography. Identity of peptides can be verified by mass spectrometry. Peptides can be solubilized in aqueous buffers at neutral pH.

The peptide agonists of the present invention may also be made by recombinant methods known in the art using both eukaryotic and prokaryotic cellular hosts.

The cyclisation of the VPAC2 receptor peptide agonists can be carried out in a solution or on a solid support. Cyclisation on a solid support can be performed immediately following solid phase synthesis of the peptide. This involves the selective or orthogonal protection of the amino acids which will be covalently linked in cyclisation.

Various preferred features and embodiments of the present invention will now be described with reference to the following non-limiting examples:

### EXAMPLE 1

### Preparation of the Selective cyclic VPAC2 Receptor Peptide Agonists by Solid Phase t-Boc Chemistry

Approximately 0.5-0.6 grams (0.35-0.45 mmole) Boc Ser(Bzl)-PAM resin is placed in a standard 60 mL reaction vessel. Double couplings are run on an Applied Biosystems ABI433A peptide synthesizer. The following side-chain protected amino acids (2 mmole cartridges of Boc amino acids) are obtained from Midwest Biotech (Fishers, Ind.) and are used in the synthesis:

Arg-tosyl (Tos), Asp-cyclohexyl ester(OcHx), Asp-9-fluorenylmethyl (Fm), Cys-p-methylbenzyl (p-MeBzl), Glu-cyclohexyl ester (OcHx), His-benzyloxymethyl(Bom), Lys-2-chlorobenzyloxycarbonyl (2Cl—Z), Lys-9-fluorenylmethoxycarbonyl (Fmoc), Orn-2-chlorobenzyloxycarbonyl (2Cl—Z), Ser-O-benzyl ether (OBzl), Thr-O-benzyl ether (OBzl), Tyr-2-bromobenzyloxycarbonyl (2Br—Z), Boc-Ser(OBzl) PAM resin, and MBHA resin. Trifluoroacetic acid (TFA), di-isopropylethylamine (DIEA), 1.0 M hydroxybenzotriazole (HOBt) in NMP and 1.0 M dicyclohexylcarbodiimide (DCC) in NMP are purchased from PE-Applied Biosystems (Foster City, Calif.). Dimethylformamide (DMF-Burdick and Jackson) and dichloromethane (DCM-Mallinkrodt) is purchased from Mays Chemical Co. (Indianapolis, Ind.). Benzotriazole-1-yl-oxy-tris-(dimethylamino)-phosphoniumhexafluorophosphate (BOP) is obtained from NovaBiochem (San Diego, Calif.).

Standard double couplings are run using either symmetric anhydride or HOBt esters, both formed using DCC. At the completion of the syntheses, the N-terminal Boc group is removed and the peptidyl resins are capped with an organic acid such as hexanoic acid using diisopropylcarbodiimide (DIC) in DMF. The resin is then treated with 20% piperidine in DMF for 20 min. The Fmoc and Fm protecting groups are selectively removed and the cyclisation is carried out by activating the aspartic acid carboxyl group with BOP in the presence of DIEA. The reaction is allowed to proceed for 24 hours and monitored by ninhydrin test. After washing with DCM, the resins are transferred to a TEFLON reaction vessel and are dried in vacuo.

Cleavages are done by attaching the reaction vessels to a HF (hydrofluoric acid) apparatus (Penninsula Laboratories). 1 mL m-cresol per gram/resin is added and 10 mL HF (purchased from AGA, Indianapolis, Ind.) is condensed into the pre-cooled vessel. 1 mL DMS per gram resin is added when methionine is present. The reactions are stirred one hour in an ice bath. The HF is removed in vacuo. The residues are suspended in ethyl ether. The solids are filtered and are washed with ether. Each peptide is extracted into aqueous acetic acid and either is freeze dried or is loaded directly onto a reverse-phase column.

Purifications are run on a 2.2×25cm VYDAC C18 column in buffer A (0.1% TFA in water). A gradient of 20% to 90% B (0.1% TFA in acetonitrile) is run on an HPLC (Waters) over 120 minutes at 10 mL/minute while monitoring the UV at 280 nm (4.0 A) and collecting one minute fractions. Appropriate fractions are combined, frozen and lyophilized. Dried products are analyzed by HPLC (0.46×15 cm METASIL AQ C18) and MALDI mass spectrometry.

Cyclic VPAC2 receptor peptide agonists with a lactam bridge linking, for example, an ornithine residue and a glutamic acid residue are prepared by selectively protecting the side chains of these residues with Fmoc and Fm, respectively. All other amino acids used in the synthesis are standard benzyl side-chain protected Boc-amino acids. Cyclisation may then be carried out immediately following solid phase synthesis of the peptide.

### EXAMPLE 2

### Preparation of the Selective VPAC2 Receptor Cyclic Peptide Agonists by Solid Phase Fmoc Chemistry

Approximately 114 mg (50 mmole) Fmoc-Ser(tBu) WANG resin (purchased from GlycoPep, Chicago, Ill.) is placed in each reaction vessel. The synthesis is conducted on a Rainin Symphony Peptide Synthesizer. Analogs with a C-terminal amide are prepared using 75 mg (50 μmole) Rink Amide AM resin (Rapp Polymere. Tuebingen, Germany).

The following Fmoc amino acids are purchased from GlycoPep (Chicago, Ill.), and NovaBiochem (La Jolla, Calif.): Arg-2,2,4,6,7-pentamethyldihydrobenzofuran-5-sulfonyl (Pbf), Asn-trityl (Trt), Asp-β-t-Butyl ester (tBu), Asp-β-allyl ester (Allyl), Glu-δ-t-butyl ester (tBu), Glu-δ-allyl ester (Allyl), Gln-trityl (Trt), His-trityl (Trt), Lys-t-butyloxycarbonyl (Boc), Lys-allyloxycarbonyl (Aloc), Orn-allyloxycarbonyl (Aloc), Ser-t-butyl ether (OtBu), Thr-t-butyl ether (OtBu), Trp-t-butyloxycarbonyl (Boc), Tyr-t-butyl ether (OtBu).

Solvents dimethylformamide (DMF-Burdick and Jackson), N-methyl pyrrolidone (NMP-Burdick and Jackson), dichloromethane (DCM-Mallinkrodt) are purchased from Mays Chemical Co. (Indianapolis, Ind.).

Hydroxybenzotrizole (HOBt), di-isopropylcarbodiimide (DIC), di-isopropylethylamine (DIEA), and piperidine (Pip) are purchased from Aldrich Chemical Co (Milwaukee, Wis.). Benzotriazole-1-yl-oxy-tris-(dimethylamino)-phosphoniumhexafluorophosphate (BOP) is obtained from NovaBiochem (San Diego, Calif.).

All amino acids are dissolved in 0.3 M concentration in DMF. Three hours DIC/HOBt activated couplings are run after 20 minutes deprotection using 20% Piperidine/DMF. Each resin is washed with DMF after deprotections and couplings. After the last coupling and deprotection, the peptidyl resins are washed with DCM and are dried in vacuo in the reaction vessel. For the N-terminal acylation, four-fold excess of symmetric anhydride of the corresponding acid is added onto the peptide resin. The symmetric anhydride is prepared by DIC activation in DCM. The reaction is allowed to proceed for 4 hours and monitored by ninhydrin test. The Aloc and Allyl protecting groups are selectively removed and the cyclisation is carried out by activating the aspartic acid carboxyl group with BOP in the presence of DIEA. The peptide resin is then washed with DCM and dried in vacuo.

The cleavage reaction is mixed for 2 hours with a cleavage cocktail consisting of 0.2 mL thioanisole, 0.2 mL methanol, 0.4 mL triisopropylsilane, per 10 mL TFA, all purchased from Aldrich Chemical Co., Milwaukee, Wis. If Cys is present in the sequence, 2% of ethanedithiol is added. The TFA filtrates are added to 40 mL ethyl ether. The precipitants are centrifuged 2 minutes at 2000 rpm. The supernatants are decanted. The pellets are resuspended in 40 mL ether, re-centrifuged, re-decanted, dried under nitrogen and then in vacuo. 0.3-0.6 mg of each product is dissolved in 1 mL 0.1% TFA/acetonitrile(ACN), with 20 μL being analyzed on HPLC [0.46×15cm METASIL AQ C18, 1 mL/min, 45° C., 214 nM (0.2A), A=0.1% TFA, B=0.1% TFA/50% ACN. Gradient=50% B to 90% B over 30 minutes].

Purifications are run on a 2.2×25 cm VYDAC C 18 column in buffer A (0.1% TFA in water). A gradient of 20% to 90% B (0.1% TFA in acetonitrile) is run on an HPLC (Waters) over 120 minutes at 10 mL/minute while monitoring the UV at 280 nm (4.0A) and collecting 1 minute fractions. Appropriate fractions are combined, frozen and lyophilized. Dried products are analyzed by HPLC (0.46×15 cm METASIL AQ C18) and MALDI mass spectrometry.

Cyclic VPAC2 receptor peptide agonists with a lactam bridge linking, for example, an ornithine residue and a glutamic acid residue are prepared by selectively protecting the side chains of these residues with Aloc and Allyl, respectively. All other amino acids used in the synthesis are standard t-butyl side-chain protected Fmoc-amino acids. Cyclisation may then be carried out on the solid support immediately following solid phase synthesis of the peptide.

### EXAMPLE 3

### In-Vitro Potency at Human VPAC2 Receptors

Alpha screen: Cells (CHO-S cells stably expressing human VPAC2 receptors) are washed in the culture flask once with PBS. Then, the cells are rinsed with enzyme free dissociation buffer. The dissociated cells are removed. The cells are then spun down and washed in stimulation buffer. For each data point, 50,000 cells suspended in stimulation buffer are used. To this buffer, Alpha screen acceptor beads are added along with the stimuli. This mixture is incubated for 60 minutes. Lysis buffer and Alpha screen donor beads are added and are incubated for 60 to 120 minutes. The Alpha screen signal (indicative of intracellular cAMP levels) is read in a suitable instrument (e.g. AlphaQuest from Perkin-Elmer). Steps including Alpha screen donor and acceptor beads are performed in reduced light. The EC₅₀ for cAMP generation is calculated from the raw signal or is based on absolute cAMP levels as determined by a standard curve performed on each plate. The test peptide concentrations are: 10000, 1000, 100, 10, 3, 1, 0.1, 0.01, 0.003, 0.001, 0.0001 and 0.00001 nM.

DiscoveRx: A CHO-S cell line stably expressing human VPAC2 receptor in a 96-well microtiter plate is seeded with 50,000 cells/well the day before the assay. The cells are allowed to attach for 24 hours in 200 μL culture medium. On the day of the experiment, the medium is removed. Also, the cells are washed twice. The cells are incubated in assay buffer plus IBMX for 15 minutes at room temperature. Afterwards, the stimuli are added and are dissolved in assay buffer. The stimuli are present for 30 minutes. Then, the assay buffer is gently removed. The cell lysis reagent of the DiscoveRx cAMP kit is added. Thereafter, the standard protocol for developing the cAMP signal as described by the manufacturer is used (DiscoveRx Inc., USA). EC₅₀ values for cAMP generation are calculated from the raw signal or are based on absolute cAMP levels as determined by a standard curve performed on each plate. The typically tested concentrations of peptide are: 1000, 300, 100, 10, 1, 0.3, 0.1, 0.01, 0.001, 0.0001 and 0 nM.

### EXAMPLE 4

### Selectivity

Binding assays: Membrane prepared from a stable VPAC2 cell line (see Example 3) or from cells transiently transfected with human VPAC1 or PAC1 are used. A filter binding assay is performed using 125I-labeled PACAP-27 for VPAC1, VPAC2 and PAC1 as the tracer.

For this assay, the solutions and equipment include:

Presoak solution: 0.5% Polyethyleneamine in Aqua dest

Buffer for flushing filter plates: 25 mM HEPES pH 7.4

Blocking buffer: 25 mM HEPES pH 7.4; 0.2% protease free BSA

Assay buffer: 25 mM HEPES pH 7.4; 0.5% protease free BSA

Dilution and assay plate: PS-Microplate, U form

Filtration Plate: Multiscreen FB Opaque Plate; 1.0 μM Type B Glasfiber filter

In order to prepare the filter plates, the presoak solution is aspirated by vacuum filtration. The plates are flushed twice with 200 μL flush buffer. 200 μL blocking buffer is added to the filter plate. The filter plate is then incubated with 200 μL presoak solution for 1 hour at room temperature.

The assay plate is filled with 25 μL assay buffer, 25 μL membranes (2.5 μg) suspended in assay buffer, 25 μL agonist in assay buffer, and 25 μL tracer (about 40000 cpm) in assay buffer. The filled plate is incubated for 1 hour with shaking.

The transfer from assay plate to filter plate is conducted. The blocking buffer is aspirated by vacuum filtration and washed two times with flush buffer. 90 μL is transferred from the assay plate to the filter plate. The 90 μL transferred from assay plate is aspirated and washed three times with 200 μL flush buffer. The plastic support is removed. It is dried for 1 hour at 60° C. 30 μL Microscint is added. The count is performed.

### EXAMPLE 5

### In vitro Potency at Rat VPAC1 and VPAC2 Receptors

DiscoveRx: CHO-PO cells are transiently transfected with rat VPAC1 or VPAC2 receptor DNA using commercially available transfection reagents (Lipofectamine from Invitrogen). The cells are seeded at a density of 10,000/well in a 96-well plate and are allowed to grow for 3 days in 200 mL culture medium. At day 3, the assay is performed.

On the day of the experiment, the medium is removed. Also, the cells are washed twice. The cells are incubated in assay buffer plus IBMX for 15 minutes at room temperature. Afterwards, the stimuli are added and are dissolved in assay buffer. The stimuli are present for 30 minutes. Then, the assay buffer is gently removed. The cell lysis reagent of the DiscoveRx cAMP kit is added. Thereafter, the standard protocol for developing the cAMP signal as described by the manufacturer is used (DiscoveRx Inc., USA). EC₅₀ values for cAMP generation are calculated from the raw signal or are based on absolute cAMP levels as determined by a standard curve performed on each plate. The typically tested concentrations of peptide are: 1000, 300, 100, 10, 1, 0.3, 0.1, 0.01, 0.001, 0.0001 and 0 nM.

### EXAMPLE 6

### In Vivo Assays

Intravenous glucose tolerance test (IVGTT): Normal Wistar rats are fasted overnight and are anesthetized prior to the experiment. A blood sampling catheter is inserted into the rats. The agonist is given subcutaneously, normally 24 h prior to the glucose challenge. Blood samples are taken from the carotid artery. A blood sample is drawn immediately prior to the injection of glucose along with the agonist. After the initial blood sample, glucose mixed is injected intravenously (i.v.). A glucose challenge of 0.5 g/kg body weight is given, injecting a total of 1.5 mL vehicle with glucose and agonist per kg body weight. The peptide concentrations are varied to produce the desired dose in μg/kg. Blood samples are drawn at 2, 4, 6 and 10 minutes after giving glucose. The control group of animals receives the same vehicle along with glucose, but with no agonist added. In some instances, 20 and 30 minute post-glucose blood samples were drawn. Aprotinin is added to the blood sample (250-500 kIU/ml blood). The plasma is then analyzed for glucose and insulin using standard methodologies.

The assay uses a formulated and calibrated peptide stock in PBS. Normally, this stock is a prediluted 100 μM stock. However, a more concentrated stock with approximately 1 mg agonist per mL is used. The specific concentration is always known. Variability in the maximal response is mostly due to variability in the vehicle dose. Protocol details are as follows:

| | |
|---|---|
| SPECIES/STRAIN/WEIGHT | Rat/Wistar Unilever/approximately 275-300 g |
| TREATMENT DURATION | Single dose |
| DOSE VOLUME/ROUTE | 1.5 mL/kg/iv |
| VEHICLE | 8% PEG300, 0.1% BSA in water |
| FOOD/WATER REGIMEN | Rats are fasted overnight prior to surgery. |
| LIVE-PHASE PARAMETERS | Animals are sacrificed at the end of the test. |
| IVGTT: Performed on rats (with two | Glucose IV bolus: 500 mg/kg as 10% |
| catheters, jugular vein and carotid | solution (5 mL/kg) at time = 0. |
| artery) of each group, under | Compound iv: 0-240 min prior to glucose |
| pentobarbital anesthesia. | Blood samplings (300 μL from carotid artery; |
| | EDTA as anticoagulant; aprotinin and PMSF |
| | as antiproteolytics; kept on ice): 0, 2, 4, 6, and 10, |
| | 20 and 30 minutes. |
| | Parameters determined: Insulin + glucose |
| TOXICOKINETICS | Plasma samples remaining after insulin |
| | measurements are kept at −20° C. and |
| | compound levels are determined. |

### EXAMPLE 7

### Rat Serum Stability Studies

In order to determine the stability of VPAC2 receptor peptide agonists in rat serum, CHO-VPAC2 cells clone #6 (96 well plates/50,000 cells/well and 1 day culture), PBS 1× (Gibco), the peptides for the analysis in a 100 μM stock solution, rat serum from a sacrificed normal Wistar rat, aprotinin, and a DiscoveRx assay kit are obtained. The rat serum is stored at 4° C. until use and is used within two weeks.

On Day 0, two 100 μL aliquots of 10 μM peptide in rat serum are prepared by adding 10 μL peptide stock to 90 μL rat serum for each aliquot. 250 kIU aprotinin/mL is added to one of these aliquots. The aliquot is stored with aprotinin at 4° C. The aliquot is stored without aprotinin at 37° C. The aliquots are incubated for 24 hours.

On Day 1, after incubation of the aliquots prepared on day 0 for 24 hours, an incubation buffer containing PBS+1.3 mM CaCl₂, 1.2 mM MgCl₂, 2 mM glucose, and 0.5 mM IBMX is prepared. A plate with 11 serial 3× dilutions of peptide in serum for the 4° C. and 37° C. aliquot is prepared for each peptide studied. 4000 nM is used as the maximal concentration. The plate(s) with cells are washed twice in incubation buffer and the cells are incubated in 50 μL incubation media per well for 15 minutes. 50 μL solution per well is transferred to the cells from the plate prepared with 11 serial 3× dilutions of peptide for the 4° C. and 37° C. aliquot for each peptide studied, using the maximal concentrations that are indicated by the primary screen, in duplicate. This step dilutes the peptide concentration by a factor of two. The cells are incubated at room temperature for 30 minutes. The supernatant is removed. 40 μL/well of the DiscoveRx antibody/extraction buffer is added. The cells are incubated on the shaker (300 rpm) for 1 hour. Normal procedure with the DiscoveRx kit is followed. cAMP standards are included in column 12. EC₅₀ values are determined from the cAMP assay data. The remaining amount of active peptide is estimated by the formula EC_{50, 4C}/EC_{50,37C} for each condition.

Other modifications of the present invention will be apparent to those skilled in the art without departing from the scope of the invention.

1. A cyclic VPAC2 peptide receptor agonist comprising the amino acid sequence.
(SEQ ID NO: 1)
C6-HSDAVFTENY(OMe)TOrnLRAibQNleAAKOrnYLNELOrnOrnGG
PSSGAPPPS.

2. A pharmaceutical compositions comprising a cyclic VPAC2 receptor peptide agonist according to claim 1 and one or more pharmaceutically acceptable diluents, carriers or excipients.

3-5. (canceled)

6. A method of treating non-insulin-dependent diabetes or insulin-dependent diabetes, or of suppressing food intake in a patient in need thereof, comprising administering to said patient an effective amount of a cyclic VPAC2 receptor peptide agonist according to claim 1.

7-8. (canceled)


SEQUENCE LISTING (7 sequences)

SEQ ID NO: 1 - protein, 39 aa, Artificial sequence
  Synthetic sequence Cyclic VPAC2 peptide receptor agonist P403
HSDAVFTENYTXLRXQXAAKXYLNELXXGGPSSGAPPPS

SEQ ID NO: 2 - protein, 28 aa, Homo sapiens
HSDAVFTDNYTRLRKQMAVKKYLNSILN

SEQ ID NO: 3 - protein, 27 aa, Homo sapiens
  MOD_RES (27)..(27): AMIDATION
HSDGIFTDSYSRYRKQMAVKKYLAAVL

SEQ ID NO: 4 - protein, 37 aa, Homo sapiens
  MOD_RES (37)..(37): AMIDATION
HSDGIFTDSYSRYRKQMAVKKYLAAVLGKRYQRVKNK

SEQ ID NO: 5 - protein, 9 aa, Artificial sequence
  Synthetic sequence N-terminal modification
SWCEPGWCR

SEQ ID NO: 6 - protein, 39 aa, Artificial sequence
  Synthetic sequence Reference peptide used for percentage sequence identity calculations
HSDAVFTENYTKLRKQXAAKKYLNDLKKGGPSSGAPPPS

SEQ ID NO: 7 - protein, 11 aa, Heloderma suspectum
GGPSSGAPPPS